(19) United States
(12) Patent Application Publication
(10) Pub. No.: US 2022/0000469 A1
(43) Pub. Date: Jan. 6, 2022

(54) APPARATUS AND SYSTEM FOR ABSORBABLE SURGICAL BUTTON AND METHODS THEREOF

(71) Applicant: Jeffrey P. Norris, Deerfield, IL (US)

(72) Inventor: Jeffrey P. Norris, Deerfield, IL (US)

(21) Appl. No.: 17/366,859

(22) Filed: Jul. 2, 2021

Related U.S. Application Data
(60) Provisional application No. 63/047,499, filed on Jul. 2, 2020.

(51) Int. Cl. A61B 17/04; A61B 17/06; A61B 17/42

(52) U.S. Cl. CPC A61B 17/0401; A61B 2017/00004; A61B 17/42; A61B 17/06166

(57) ABSTRACT

An absorbable surgical button, system and methods that relate to an absorbable surgical button, made to dissolve over time, along with related insertion or pusher tools and components, such as a button pusher tool, to facilitate sacrospinous fixation surgical procedures for vaginal prolapse through a vaginal approach or other similar surgeries, by simplifying the advancement and placement of the absorbable surgical button.

The present application claims priority to U.S. Provisional Patent Application Ser. No. 63/047,499, filed Jul. 2, 2020, entitled “Apparatus And System For Absorbable Surgical Button And Method Thereof”, which is hereby incorporated by reference in its entirety.

### a. TECHNICAL FIELD

The present disclosure relates to an absorbable or dissolvable surgical button apparatus and systems, along with the methods for using and securing the absorbable surgical button during sacrospinous fixation surgical treatment for vaginal prolapse through a vaginal approach or other similar surgeries. In particular, the present disclosure relates to absorbable surgical buttons, made to dissolve over time, along with related components, such as a button insertion or pusher tool, to facilitate the surgical procedure by simplifying the placement of the absorbable surgical button.

The present disclosure relates to surgical procedures using a surgical button and related components during sacrospinous fixation surgical procedures for vaginal prolapse through a vaginal approach or other similar surgeries. In the preferred embodiment and as referenced herein, the surgical button is absorbable or dissolvable wherein the absorbable surgical button and possibly any sutures are absorbed or dissolved over time so as to not remain in the patient's body, thereby improving the healing process.

The present disclosure further relates to absorbable surgical buttons and systems that facilitate the surgical process during sacrospinous fixation surgical procedures by increasing the surgeon's visibility during the procedure thereby simplifying the procedure, and increasing the surgeon's ability to reliably secure the apex of the patient's vagina to the sacrospinous ligament thereby reducing the time needed for the procedure. These advantages mitigate or reduce instances of patient trauma, risk of infection, patient pain and the potential need for follow up surgeries. The advantages of the surgical procedures described herein can more often be accomplished as out-patient procedures.

The present disclosure further relates to absorbable surgical buttons and systems that facilitate the surgical process during sacrospinous fixation surgical wherein the absorbable surgical buttons are also impregnated or permeated with medicine or medicinal properties such that as the surgical buttons are absorbed, they provide medication to the patient and to the location of the surgery to assist the healing process.

The present disclosure contemplates that each of the above-referenced absorbable surgical buttons and systems can be incorporated into current prolapse surgeries without the need for additional steps, additional equipment or additional time, thereby allowing for quicker procedures and reducing the chance of surgical complications or failures.

### b. BACKGROUND OF DISCLOSURE

In medicine, prolapse is a condition in which an organ falls down or slips out of place. It is often used to describe organs protruding through the vagina, rectum, or for the misalignment of heart valves.

Along those lines, pelvic organ prolapse (POP) is characterized by the descent of pelvic organs from their normal positions. In women, the condition usually occurs when the pelvic floor or pelvic diagram collapses after gynecological cancer treatment, childbirth or heavy lifting. The pelvic floor, which is composed of muscle fibers and connective tissue, spans across the bony structures of the pelvis and separates the pelvic cavity above from the perineal region below. To accommodate the birth canal, a female's pelvic cavity is larger than a male's pelvic cavity. As such, the pelvic floor tends to be considered a part of female anatomy, but males have an equivalent pelvic floor. For example, sling procedures in men are used in the treatment of stress urinary incontinence, which can be a common condition after undergoing prostate surgery.

The injury occurs to fascia membranes and other connective structures and can result in numerous types of POP. In cystocele and urethrocele prolapse, both types of anterior vaginal wall prolapse, the bladder or urethra protrude into the vagina, respectively. In rectocele and enterocele prolapse, types of posterior wall prolapse, the rectum and small intestine protrudes into the vagina, respectively. In vaginal vault and uterine prolapses, both types of apical vaginal prolapse, the roof of the vagina is weakened, often after a hysterectomy, and the uterus protrude into the vagina. Treatment for these different types of prolapse can involve dietary and lifestyle changes, physical therapy, or surgery.

In women, vaginal prolapse or uterine prolapse may occur when pelvic ligaments and supportive structures are weakened. One surgical treatment is sacrospinous fixation. In this surgery, the apex of the vagina is sutured to the sacrospinous ligament, or another structurally supportive member within the pelvic region, which may offer a sturdier support than weakened pelvic ligaments, ideally preventing further prolapse. Since most anterior and posterior prolapses are caused by apical support defects, supporting the vaginal apex region may relieve most of the apically related prolapses.

Although there are numerous devices and procedures used for the above-referenced procedures, usually these surgical procedures include placing an implant, such as a mesh or graft, within the pelvic region, which is implanted into the pelvic region through one or more vaginal incisions or through external and/or transabdominal incisions. Correct and secure placement can be challenging due to limited intra-operative exposure. Additionally, these implants are left inside the body and remain even after the pelvic region has healed and is self-supported.

In order to provide a system and method for transvaginal sacrospinous ligament fixation, one solution is directed to the Anchor Delivery System And Method disclosed and claimed in U.S. Pat. No. 10,299,828, to Goldberg et al., which discloses a system for incisionless transvaginal sacrospinous ligament fixation, including an anchoring unit configured to affix the vaginal wall to the sacrospinous ligament, and to a piercing tip configured to pierce the vaginal wall, pierce the sacrospinous ligament, and deploy a portion of the anchoring unit through the pierced vaginal wall and the pierced sacrospinous ligament, thereby disposing said first portion at said sacrospinous ligament.

Another example of a prolapse treatment is directed to the Pelvic Floor Treatments And Related Tools And Implants disclosed and claimed in U.S. Pat. No. 10,010,394, to Khamis et al., which discloses implants, tools, and methods useful for treating pelvic conditions such as prolapse, and discloses placing the implant to support pelvic tissue. The implants, tools, and methods involve one or more of an insertion tool that works in coordination with a sheath, adjusting engagements, specific implants and pieces of implants, for placement of implants at locations within the pelvic region, and insertion, adjusting, and grommet management tools.

Another example of a method for treating prolapse conditions is the Method for treatment of pelvic organ prolapse conditions, U.S. patent application Ser. No. 13/157,564, to Arnold, which discloses a method for treating pelvic organ prolapse conditions via a vaginal approach, including identifying the presumptive apex of the vagina and the ischial spine; pressing the presumptive apex of the vagina onto the ischial spine; while maintaining contact between the presumptive apex of the vagina and the sacrospinous ligament. Continuously sweeping the presumptive apex of the vagina in a dorso-medial direction from the ischial spine along the sacrospinous ligament to a first location approximately two centimeters from the ischial spine, and while maintaining contact between the presumptive apex of the vagina and the sacrospinous ligament, attaching the presumptive apex to the sacrospinous ligament at the first location using a first tissue anchor.

These examples disclose apparatus, systems and methods for repairing or attempting to repair prolapse conditions. However, none of these structures or any other that Applicant is currently aware of, provide the use of an absorbable surgical button apparatus, system, or methods for use in a sacrospinous fixation surgical treatment for vaginal prolapse through a vaginal approach.

As such, there is a need for an absorbable surgical button apparatus, and related components and systems, along with the methods for implanting and using the absorbable surgical buttons, in surgical treatment for sacrospinous fixation for vaginal prolapse conditions. There is also a need for a surgical procedure that improves upon the method of securing the vaginal apex to the point of fixation. This can be accomplished with a method that incorporates an absorbable surgical button that will dissolve after the patient has healed and after the healed area can provide the necessary support on its own. Additionally, there are no such devices that are combined with a medicinal property such that, upon dissolving, the properties of the absorbable button will assist in the healing process.

The foregoing is intended only to illustrate the present technical field and background art and should not be taken as a limitation or disavowal of claim scope.

### BRIEF SUMMARY OF THE DISCLOSURE

The present disclosure relates to an absorbable surgical button apparatus, related components and systems, along with the methods for using and securing the absorbable surgical buttons during sacrospinous fixation surgery for vaginal prolapse through or via a vaginal approach.

The present disclosure relates to an absorbable surgical buttons, related components and systems to facilitate the surgical process by increasing the surgeon's visibility of the surgical location during the procedure and to provide structural support during the healing process, and thereafter dissolve so as to not remain in the patient's body when that structural support is no longer needed.

The present disclosure relates to a method for using the absorbable surgical button, and related components, such as a button pusher tool, along with systems to facilitate the implanting of the absorbable surgical button during sacrospinous fixation surgical procedures via vaginal methodology by increasing the surgeon's visibility of the location of the implant during the procedure to allow for the simplification of the procedure, thereby reducing the time needed for the surgery or procedure, thereby reducing the patient's trauma and the patient's risk of infection, along with a similar reduction of the patient's pain and a reduction of the potential need for follow up surgeries.

The present disclosure further relates to absorbable surgical buttons and systems that facilitate the surgical process by providing an absorbable button that is impregnated or permeated with medicine such that as the surgical button is absorbed and dissolves, it provides medication to the patient to assist the healing process.

The present disclosure contemplates that each of the above-referenced absorbable surgical buttons, along with the related components for properly and easily inserting, advancing or installing the absorbable buttons and systems, can be incorporated into current prolapse surgeries without additional surgical steps or additional equipment thereby allowing for quicker surgical procedures and thereby reducing the chance of errors during the surgical procedures.

The present disclosure contemplates that the absorbable surgical buttons, along with the related components for properly and easily inserting, advancing or installing the absorbable buttons and systems, and related techniques, may be able to replicate improved outcomes as seen in open and robotic sacrocolpopexy procedures with less risk and quicker recovery times.

Additional objectives and advantages of the present disclosure will become apparent to one having ordinary skill in the art after reading the specification in light of the drawing figures, however, the spirit and scope of the present invention should not be limited to the description of the embodiments contained herein.

### BRIEF DESCRIPTION OF THE DRAWINGS

FIG. 1 is a perspective view of an absorbable surgical button apparatus and pusher tool or component in accordance with the present disclosure.

FIG. 2 is a front view of portions of a human body showing the relationship between the various ligaments of the pelvic region.

FIG. 3 is a perspective view the female pelvic muscles.

FIGS. 4A and 4B are perspective views of a vaginal prolapse.

FIG. 5 is a perspective view of an absorbable surgical button being used to repair a vaginal prolapse during a surgical procedure in accordance with the present disclosure.

FIGS. 6A and 6B are perspective views of an absorbable surgical button after repairing a vaginal prolapse in accordance with the present disclosure.

FIGS. 7A and 7B are perspective views of an absorbable surgical button immediately after repairing a vaginal prolapse (7A), and then a period of time later (7B), in accordance with the present disclosure.

### DETAILED DESCRIPTION OF THE DISCLOSURE

As stated herein, the objective of the present disclosure is to provide an improved absorbable surgical button and related components, an improved absorbable surgical button system, along with improved methods for inserting, advancing or installing the absorbable surgical button to alleviate pelvic organ or vaginal prolapse.

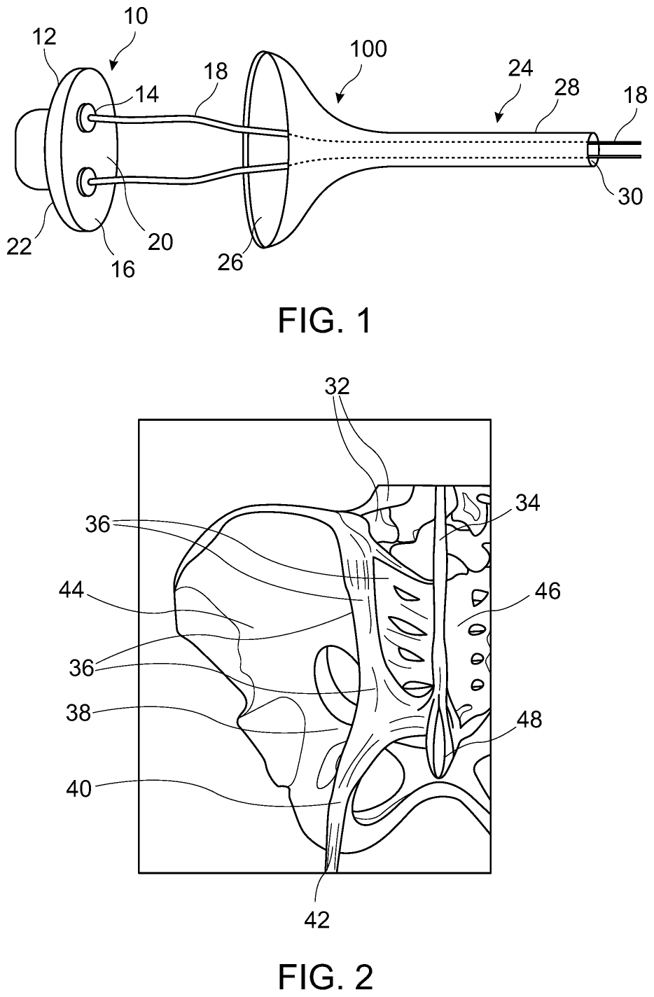

Referring to the drawings, wherein like reference numerals refer to the same or similar features in the various views, FIGS. 1 and 5 through 7A show different views of the improved absorbable surgical button 10 in the preferred embodiment. FIG. 1 shows a perspective view of the absorbable surgical button 10 of the present disclosure, which in the preferred embodiment comprises solid, circular-shaped, button-like configuration 12 having at least one button hole 14 in the interior portion 16, and in the preferred embodiment, two button holes 14 in the interior portion 16 of the absorbable surgical button 10. In alternative embodiments, there may more than two button holes 14 and the absorbable surgical button 10 may be other shapes, for example oval or rectangular, for optimal functionality, as described herein.

In the preferred embodiment, the absorbable surgical button 10 is circular-shaped and approximately 1 inch in diameter. Different materials and different sizes and shapes of absorbable surgical buttons or absorbable buttons 10 can be used, including synthetics, depending on the location of the attachment to the sacrospinous ligament, another ligament, or other parameters. Additionally, in an embodiment, the absorbable surgical buttons 10 for facilitating the surgical process can be impregnated or permeated with medicine as understood by those having ordinary skill in the art such that as the surgical button 10 is absorbed and dissolves, it can provide medication to the patient to assist in the healing process.

In use, the absorbable surgical button 10 will be held in place using one or more sutures 18, either individually tied through each of the button holes 14 in the absorbable surgical button 10, or as a single suture 18 threaded through multiple button holes 14. The absorbable surgical button 10 will have a first side 20 and a second side 22, with the second side 22 facing the sacrospinous ligament during the surgical procedure, as described herein.

Additionally, the absorbable surgical button system 100 comprises a button pusher or insertion tool 24, used for advancing or installing the absorbable surgical button 10 into the vagina during the prolapse repair surgery, as described herein. The button pusher tool 24 comprises a button holder area 26 at the distal end, which is shaped and sized to accept and temporarily secure the absorbable surgical button 10 during the surgical procedure for proper placement while the surgeon ties the sutures 18 to hold the absorbable surgical button 10 in the correct location.

The button pusher tool 24 further comprises a conduit 28, which can be cylindrical and may comprise a hollow channel 30. The conduit 28 provides a handle (not shown) for the surgeon to hold the button pusher tool 24, which in turn holds the absorbable surgical button 10, with the second side of the absorbable surgical button 10 away from the surgeon. The hollow channel 30 provides a conduit for the surgeon to access the sutures 18, in the preferred embodiment, for securing the absorbable surgical button 10 in the proper location.

FIG. 2 shows a front facing view of portions of the human anatomy showing the spatial relationship between various ligaments located in the pelvic region. Ligaments are short bands of tough, flexible, fibrous connective tissue that connect bones, cartilage or joints, or a combination of those together. In particular, the function of the ligaments is to keep the structure stable and to prevent movement of bones, cartilage and joints.

There are a number of ligaments in the pelvic region, including the iliolumbar ligament 32, the supraspinous ligament 34, the posterior sacro-iliac ligaments 36, the sacrospinous ligament 38, the sacrotuberous ligament 40, and the tendon of long head of biceps femoris muscle 42. In particular, the function of these ligaments is to keep the pelvic structure stable and to prevent unwanted movement of the bones, cartilage and joints of the pelvic region.

There are also a number of bones in the pelvic region at which the ligaments are attached to support the bone structure and keep everything in the proper location. A few of these bones include the ilium 44, the sacrum 46 and the coccyx 48.

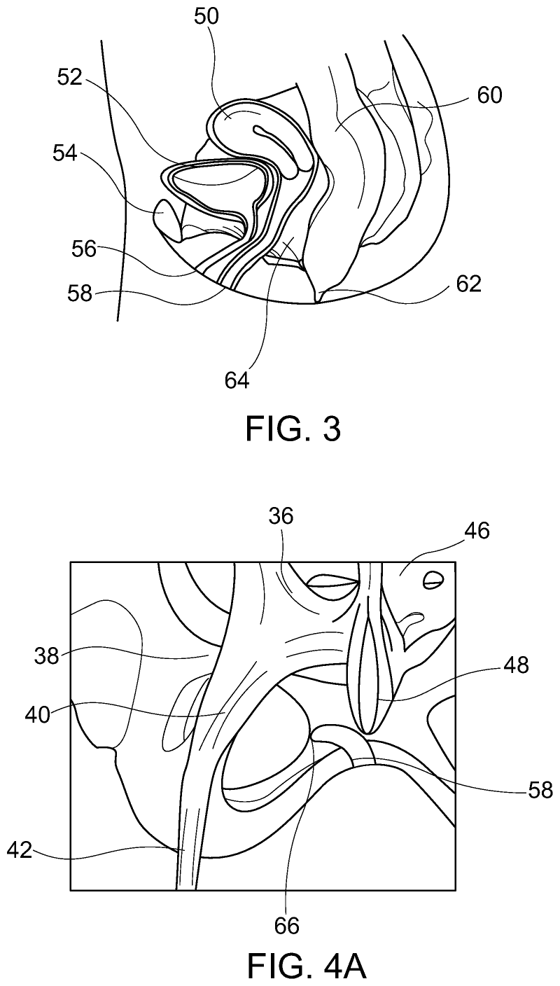

Additionally, the female anatomy differs greatly from the male anatomy, especially in the pelvic region. For spatial reference, FIG. 3 shows some of the anatomy of the female pelvic region, including certain muscles and organs. In particular, FIG. 3 shows the uterus 50, bladder 52, pubic bone 54, urethra 56, vagina 58, rectum 60, anus 62 and pelvic floor muscle 64. As described herein and as an example, when a pelvic organ prolapse occurs, such as cystocele prolapse, a type of anterior vaginal wall prolapse, the bladder 52 protrudes into the vagina 58.

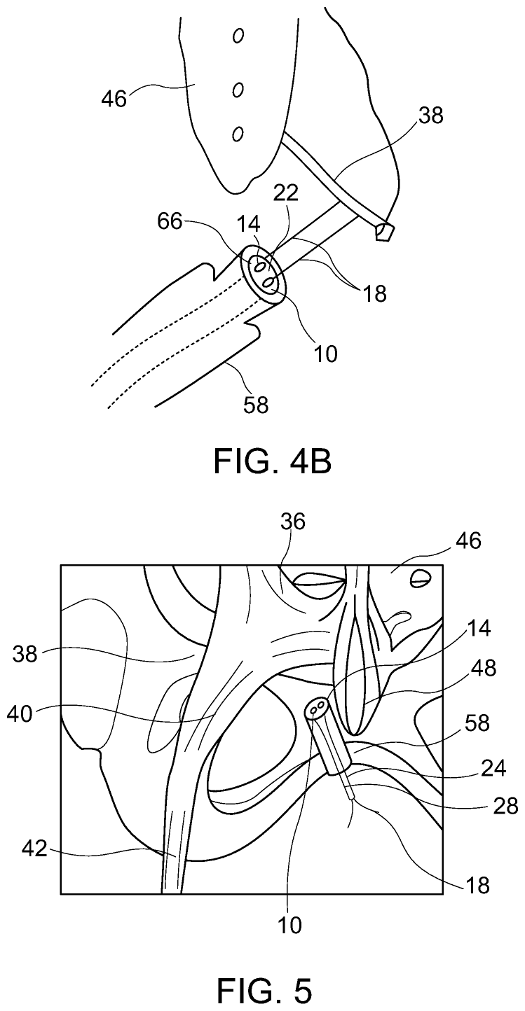

FIGS. 4A and 4B are perspective views of a vaginal prolapse in which the tissues that allow for proper vaginal support in the pelvis have been compromised thereby creating a condition in which some of the above-referenced organs may compress the vaginal walls and protrude through the vaginal vault and into the vagina 58. The sacrospinous fixation surgical procedure described herein, where the distal end 66 of the vagina 58 is surgically attached to the sacrospinous ligament 38 is used to repair vaginal prolapse.

FIG. 5 shows the absorbable surgical button system 100 in use during fixation surgery to repair a vaginal prolapse in accordance with the present disclosure. During the surgery, the absorbable surgical button 10 is held secure by the button holder area 26 of the button pusher tool 24. The surgeon then advances the distal end of the button pusher tool 24 into and towards the distal end 66 of the vagina 58.

Once the surgeon reaches the distal end 66 of the vagina 58 with the absorbable surgical button 10, the vagina 58 can be properly positioned against the sacrospinous ligament 38. At this point, the second side 22 of the absorbable button 10 will press the distal end 66 of the vagina 58 against the sacrospinous ligament 38.

In an embodiment, the surgeon makes an incision from inside the vagina 58 and attaches one or more sutures 18 to the sacrospinous ligament 38 through the distal end 66 of the vagina 58. The surgeon then advances the free end(s) of the one or more sutures 18 through the vaginal apex 58 and through the one or more holes 14 in the absorbable surgical button 10. The vaginal incision is then closed.

The surgeon can then use the button pusher tool 24 with the sutures 18 threaded through the hollow channel 30 in the cylindrical conduit 28. This will allow the absorbable surgical button 10 to be properly positioned in the distal end 66 of the vagina 58 using the button pusher tool 24. Once properly positioned, the button pusher tool 24 can be removed and the sutures can be tied to keep the absorbable surgical button 10 in place holding the distal end 66 of the vagina 58 against the sacrospinous ligament 38 while the patient heals.

Alternative embodiments include anchoring the sutures 18 or another type of anchor device into the sacrospinous ligament 38 and then advances the hanging suture ends through the distal end 66 of the vagina 58 to be connected to the absorbable surgical button 10. Additionally, the absorbable surgical button 10 may have an anchor attached thereto to be able to be placed into the distal end 66 of the vagina 58 and then stapled or otherwise connected through the distal end 66 of the vagina 58 to the sacrospinous ligament 38.

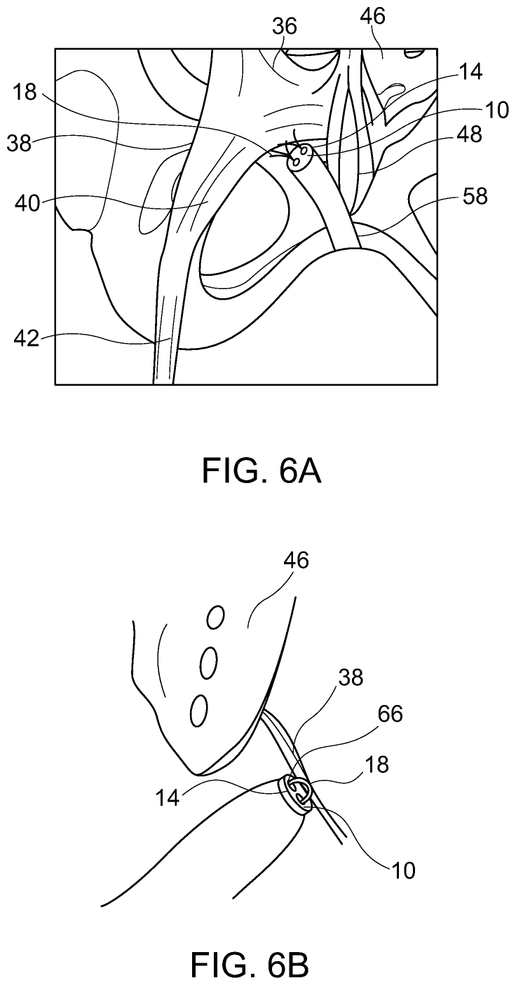

FIGS. 6A and 6B show the absorbable surgical button 10 attaching the distal end 66 of the vagina 58 to the sacrospinous ligament 38. At this time, the absorbable surgical button 10 will continue to hold the distal end 66 of the vagina 58 to the sacrospinous ligament 38 while the vagina 58 begins to heal and hold in place over time. As this occurs, the absorbable surgical button 10 begins to dissolve so that it dissolves after it is no longer needed to hold the vagina 58 in the proper location. Additionally, the sutures 18 may also be of the dissolving type, such that after neither the absorbable surgical button 10 nor the sutures 18 are necessary, they have dissolved.

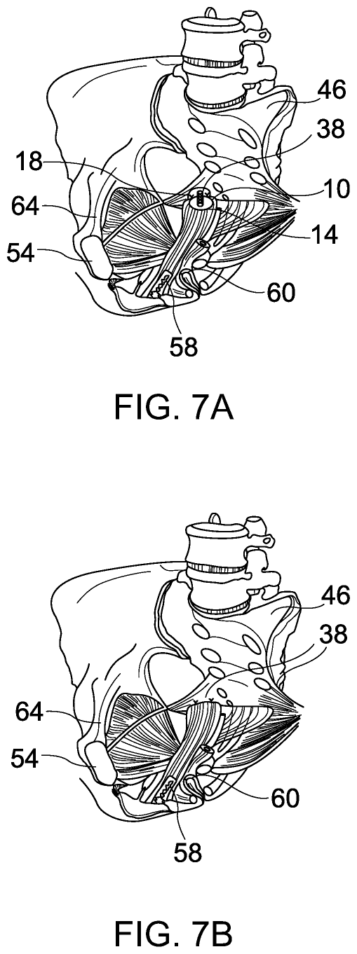

FIG. 7A shows a perspective view of an absorbable surgical button 10 properly positioned in relation to the vagina 58 and the sacrospinous ligament 38 after repairing a vaginal prolapse in accordance with the present disclosure. The sutures 18, which are advanced through the button holes 14 in the absorbable surgical button 10, hold the distal end 66 of the vagina 58 to the sacrospinous ligament 38. At a later time, FIG. 7B shows that both the absorbable surgical button 10 and the sutures 18 have dissolved, leaving only the distal end 66 of the vagina 58 attached to the sacrospinous ligament 38.

The sacrospinous fixation surgical treatment or procedure for vaginal prolapse described herein can also be applied to other similar surgeries, such as ileococcygeus suspension procedure, or others.

As a non-limiting example, the absorbable surgical button 10 may also be used to maintain tension following sling procedures used in the treatment of male stress incontinence. This is a common condition in men who have undergone prostate surgery. Following placement of the sling against the bulbar urethra (not shown), tension is applied on the arms of the sling in order to compress the urethra, thereby restoring continence. The absorbable surgical button 10 could be utilized to maintain tension while the sling heals in place. Currently, there is no efficient method for accomplishing this goal.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment.

Further, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation given that such combination is not illogical or non-functional. Although numerous embodiments of this invention have been described above with a certain degree of particularity, those skilled in the art could make numerous alterations to the disclosed embodiments without departing from the spirit or scope of this disclosure.

All directional references (e.g., plus, minus, upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the any aspect of the disclosure.

As used herein, the phrased “configured to,” “configured for,” and similar phrases indicate that the subject device, apparatus, or system is designed and/or constructed (e.g., through appropriate hardware, software, and/or components) to fulfill one or more specific object purposes, not that the subject device, apparatus, or system is merely capable of performing the object purpose. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A surgical button system for repairing a patient's vaginal prolapse, comprising:
A button apparatus, said button apparatus comprising at least one button apparatus hole in an interior portion of said button apparatus, said button apparatus having a first side and a second side;
a button apparatus pusher tool, said button apparatus pusher tool comprising a button apparatus holder area at a distal end and a conduit, said button apparatus holder area configured to accept and temporarily secure said button apparatus such that when accepted and secured, said second side of said button apparatus is facing away from said distal end of said button apparatus pusher tool;
at least one suture, said at least one suture configured to fit through said at least one button apparatus hole, wherein when said button apparatus is accepted and secured to said button apparatus holder area and said distal end of said button apparatus pusher tool is advanced into a patient's vagina and to the distal end of said patient's vagina, the second side of said button apparatus can be attached to a sacrospinous ligament by suturing the button apparatus to and through the distal end of the vagina and to the sacrospinous ligament.

2. The surgical button system for repairing a patient's vaginal prolapse of claim 1, wherein said button apparatus is absorbable such that said button apparatus will dissolve.

3. The surgical button system for repairing a patient's vaginal prolapse of claim 1, wherein said at least one suture is absorbable such that said at least one suture will dissolve.

4. The surgical button system for repairing a patient's vaginal prolapse of claim 1, wherein said at least one button apparatus hole comprises two holes.

5. The surgical button system for repairing a patient's vaginal prolapse of claim 1, wherein said button apparatus is a circular shape.

6. The surgical button system for repairing a patient's vaginal prolapse of claim 1, wherein said button apparatus is an oval shape.

7. The surgical button system for repairing a patient's vaginal prolapse of claim 1, wherein said conduit is cylindrical.

8. The surgical button system for repairing a patient's vaginal prolapse of claim 1, wherein said conduit comprises a channel.

9. The surgical button system for repairing a patient's vaginal prolapse of claim 8, wherein said channel is hollow.

10. A method of implanting an absorbable surgical button apparatus for repairing a patient's vaginal prolapse, comprising a button apparatus, a button apparatus pusher tool, and at least one suture, said button apparatus being absorbable and comprising at least one button apparatus hole in an interior portion of said button apparatus, said button apparatus having a first side and a second side, said button apparatus pusher tool comprising a button apparatus holder area at a distal end and a conduit, said button apparatus holder area configured to accept and temporarily secure said button apparatus, said at least one suture configured to fit through said at least one button apparatus hole, the steps comprising:
a. securing said button apparatus to said distal end of said button apparatus pusher tool at said button apparatus holder area such that said second side of said button apparatus faces away from said button apparatus pusher tool;
b. threading said at least one suture through said conduit and through said at least one button apparatus hole;
c. inserting said distal end of said button apparatus pusher tool into a patient's vagina;
d. advancing said distal end of said button apparatus pusher tool to the distal end of said patient's vagina;
e. using the at least one suture, attaching the second side of said button apparatus through the distal end of the patient's vagina to the sacrospinous ligament;
f. allowing said button apparatus to dissolve.

11. The surgical button system for repairing a patient's vaginal prolapse of claim 10, wherein said at least one suture is absorbable such that said at least one suture will dissolve.

12. The surgical button system for repairing a patient's vaginal prolapse of claim 10, wherein said at least one button apparatus hole comprises two holes.

13. The surgical button system for repairing a patient's vaginal prolapse of claim 10, wherein said button apparatus is a circular shape.

14. The surgical button system for repairing a patient's vaginal prolapse of claim 10, wherein said button apparatus is an oval shape.

15. The surgical button system for repairing a patient's vaginal prolapse of claim 10, wherein said conduit is cylindrical.

16. The surgical button system for repairing a patient's vaginal prolapse of claim 10, wherein said conduit comprises a channel.

17. The surgical button system for repairing a patient's vaginal prolapse of claim 16, wherein said channel is hollow.
